(19) United States
(12) Patent Application Publication
(10) Pub. No.: US 2014/0155796 A1
(43) Pub. Date: Jun. 5, 2014

(54) BACK BRACE TYPE SURGERY POSITIONING APPARATUS AND NAVIGATION SYSTEM HAVING THE SAME

(71) Applicant: NATIONAL TAIWAN UNIVERSITY, Taipei (TW)

(72) Inventor: Jaw-Lin Yang, Taipei (TW)

(73) Assignee: NATIONAL TAIWAN UNIVERSITY, Taipei (TW)

(21) Appl. No.: 14/072,148

(22) Filed: Nov. 5, 2013

(30) Foreign Application Priority Data
Dec. 5, 2012 (TW) 101145590

(51) Int. Cl. A61F 5/02

(52) U.S. Cl. CPC A61F 5/02
USPC 602/19

(57) ABSTRACT

A back brace type surgery positioning apparatus is provided, which includes a back brace, a positioning mechanism and an operation-assisting member. The back brace has a main board configured to be disposed on the back of a patient, an abdomen pressing board configured to be disposed on the abdomen of the patient, a plurality of positioning members for connecting the main board and the abdomen pressing board, and a positioning base board disposed on the main board. The positioning mechanism is connected to the positioning base board and has a support rack, and first and second rotating arms pivotally mounted to and rotatable relatively to the support rack, and the second rotating arm is configured to secure a surgical tool in place. The operation-assisting member is fastened by the first and second rotating arms and configured to adjust the direction and position of the surgical tool.

### BACKGROUND OF THE INVENTION

1. Field of the Invention

The present invention relates to positioning apparatuses, and more particularly, to a back brace type surgery positioning apparatus and a navigation system applicable to minimally invasive surgery.

2. Description of Related Art

Minimally invasive surgery is classified into two categories. One is endoscopic surgery and the other is percutaneous interventional treatment. Usually, a surgeon performs a percutaneous interventional treatment through a small incision on a patient with the help of X-ray images. The treatment process completely depends on the surgeon's knowledge, judgment and experience. A non-experienced surgeon may need more X-ray images to make a judgment, thereby increasing the radiation risk to the patient and the surgeon.

Accordingly, optical navigation apparatuses have been developed to overcome the above-described drawback. In a conventional optical navigation apparatus, X-ray images are captured before a surgical operation and registered along with a mark placed on a patient through an operational system. During the surgical operation, the marks of the patient and surgical tools are tracked by the image capture device so as to obtain and display a relative positional relationship between the patient and the surgical tools, thereby facilitating the surgeon to locate the surgical field. However, the marks of the patient and the surgical tools may be blocked during the surgical operation due to unsuitable position of staff or the image capture device so as to lead to a failure in tracking the marks and consequently increasing the risk of surgery. Accordingly, a mechanical navigation system is provided. However, the position of the mark placed on the patient usually needs to be manually measured for register, thereby complicating the process. Therefore, surgeons are reluctant to use the mechanical navigation system.

In the above-described optical and mechanical navigation systems, a structural body having the mark must be connected to hard tissues such as bones around the surgical field so as to ensure that the relative positional relationship between the mark and the surgical field is not changed during the surgical operation. As such, an incision is required to achieve such a connection, thereby violating the purpose of minimally invasive surgery. Accordingly, it is proposed that the structural body having the mark can be fixed around the surgical field by flexible bands. However, such flexible bands have low stability and lead to a poor positioning effect of the structural body.

Therefore, there is a need to provide an apparatus to overcome the above-described drawbacks.

### SUMMARY OF THE INVENTION

The present invention provides a back brace type surgery positioning apparatus for mechanically positioning so as to overcome the conventional drawback of failure in tracking a surgical tool through an optical navigation system.

The present invention further provides a back brace type surgery positioning apparatus that uses a back brace as a base for a positioning mechanism so as to improve the position stability of the positioning mechanism.

The present invention further provides a navigation system having a back brace type surgery positioning apparatus so as to capture computed tomography (CT) images of a plurality of positioning-assisting units before a surgical operation to thereby achieve an improved positioning effect.

Accordingly, the present invention provides a back brace type surgery positioning apparatus, which comprises: a back brace, a positioning mechanism and an operation-assisting member. The back brace comprises a main board, an abdomen pressing board, a plurality of positioning members for connecting the main board and the abdomen pressing board, and a positioning base board disposed on the main board. The main board has a support portion, an extending portion integrally formed with the support portion, and hollow portions formed at two sides of the support portion for facilitating a surgical operation. The positioning base board is disposed on the support portion of the main board and has a plurality of positioning-assisting units. The positioning mechanism is connected to the positioning base board. The positioning mechanism comprises a support rack, and a first and a second rotating arms pivotally mounted to and rotatable with respect to the support rack and provided with a plurality of encoders for measuring displacement of the first and second rotating arms. The second rotating arm is configured to secure a surgical tool in place. The operation-assisting member is fastened by the first and second rotating arms and configured to adjust the direction and position of the surgical tool.

The present invention further provides a navigation system, which comprises a back brace type surgery positioning apparatus and a computer device. The back brace type surgery positioning apparatus comprises: a back brace, a positioning mechanism and an operation-assisting member. The back brace comprises: a main board, an abdomen pressing board, a plurality of positioning members for connecting the main board and the abdomen pressing board, and a positioning base board disposed on the main board. The main board has a support portion, an extending portion integrally formed with the support portion, and hollow portions formed at two sides of the support portion for facilitating a surgical operation. The positioning base board is disposed on the support portion of the main board and has a plurality of positioning-assisting units. The positioning mechanism is connected to the positioning base board. The positioning mechanism comprises a support rack, and first and second rotating arms pivotally mounted to and rotatable with respect to the support rack and provided with a plurality of encoders for measuring displacement of the first and second rotating arms. The second rotating arm is configured to secure a surgical tool in place. The operation-assisting member is fastened by the first and second rotating arms and configured to adjust the direction and position of the surgical tool. The computer device is configured to store a plurality of computed tomography (CT) images of the back of a patient and electrically connected to the encoders for receiving displacement data from the encoders and integrating the displacement data with the CT images to thereby display the direction and position of the surgical tool.

Therefore, the present invention provides a mechanical positioning structure to overcome the conventional failure in tracking a surgical tool through an optical navigation system. Further, the present invention achieves a preferred positioning effect by displaying the position of the positioning base board in CT images through a plurality of positioning-assisting units, thereby avoid any incision on the patient as in the prior art for positioning. Furthermore, by using the back brace made of a hard material as a base of the positioning mechanism, the present invention improves the position stability of the positioning mechanism. Moreover, the back brace is designed to comply with the curve of human body so as to cause the back brace to be closely attached to the human body, thus reducing relative movement between the back brace type surgery positioning apparatus and the human body and consequently achieving a preferred positioning effect.

### BRIEF DESCRIPTION OF DRAWINGS

FIG. 1 is a schematic view of a back brace type surgery positioning apparatus according to the present invention;

FIG. 2A is a schematic view of a back brace of a back brace type surgery positioning apparatus according to the present invention;

FIG. 2B is another schematic view of the back brace according to the present invention;

FIG. 3 is a schematic view showing an application of the back brace type surgery positioning apparatus according to the present invention; and

FIG. 4 is a block diagram of a navigation system according to the present invention.

### DETAILED DESCRIPTION OF PREFERRED EMBODIMENTS

The following illustrative embodiments are provided to illustrate the disclosure of the present invention, these and other advantages and effects can be apparent to those in the art after reading this specification.

It should be noted that all the drawings are only for illustrative purpose and not intended to limit the present invention. Various modification and variations can be made without departing from the spirit of the present invention. Further, terms such as “upper”, “lower”, “left”, “right”, “one”, “two” etc. are merely for illustrative purpose and should not be construed to limit the scope of the present invention.

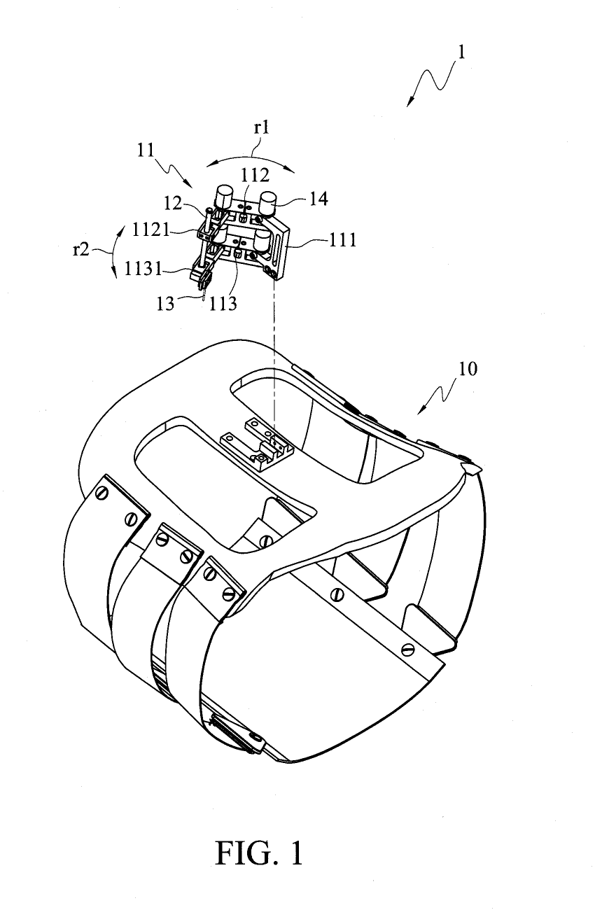

FIG. 1 is a schematic view showing a back brace type surgery positioning apparatus 1 of the present invention. Referring to FIG. 1, the back brace type surgery positioning apparatus 1 has a back brace 10, a positioning mechanism 11 connected to the back brace 10 and an operation-assisting member 12 fastened by the positioning mechanism 11. The back brace 10 is disposed on the surgical portion of the patient to serve as a base for the positioning mechanism 11 so as to improve the position stability of the positioning mechanism 11. The positioning mechanism 11 has a support rack 111, a first rotating arm 112 and a second rotating arm 113.

The first and second rotating arms 112, 113 are pivotally mounted to and rotatable with respect to the support rack 111. The operation-assisting member 12 is fastened by the first and second rotating arms 112, 113, and a surgical tool 13 is further fastened by the second rotating arm 113. The operation-assisting member 12 is operated to make the first and second rotating arms 112, 113 rotate with respect to the support rack 111 in a first rotational direction r1, thereby adjusting the position of the first and second rotating arms 112, 113 in the surgical portion. Further, the first rotating arm 112 has a first fastening member 1121, the second rotating arm 113 has a second fastening member 1131, and the operation-assisting member 12 is fastened by the first fastening member 1121 of the first rotating arm 112 and the second fastening member 1131 of the second rotating arm 113. Therefore, the operation-assisting member 12 is operated to make the first and second fastening members 1121, 1131 rotate in a rotational direction r2, thereby adjusting the direction and position of the surgical tool 13.

A plurality of encoders 14 are disposed on the first and second rotating arms 112, 113, respectively, and used for measuring displacement of the first and second rotating arms 112, 113.

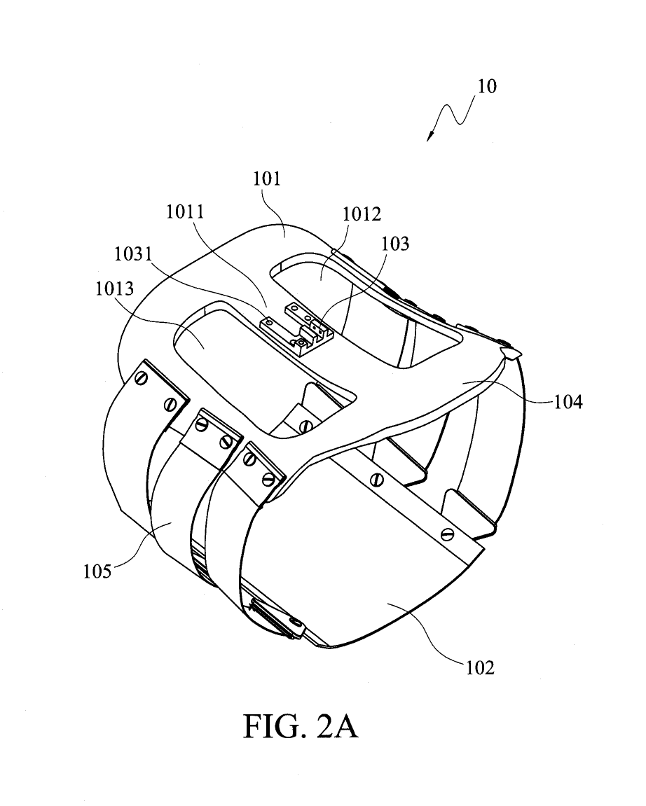

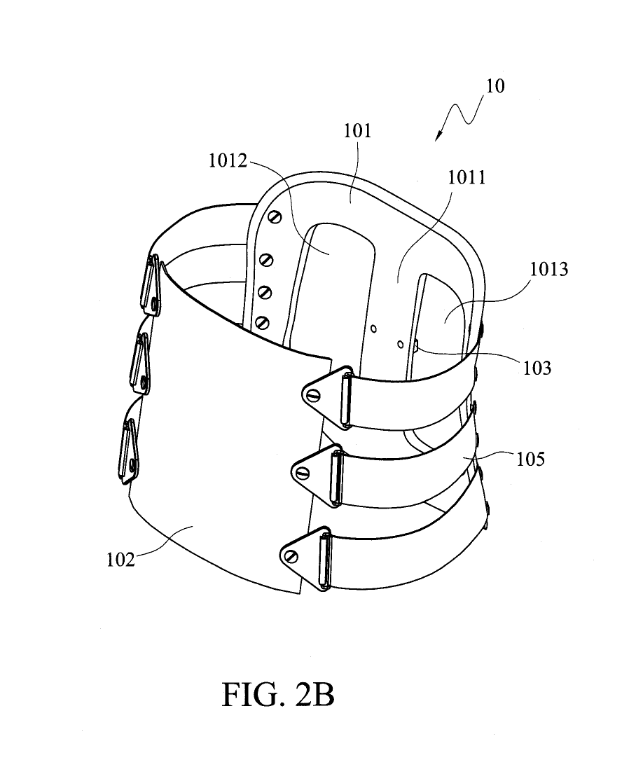

FIGS. 2A and 2B show different views of a back brace according to the present invention. Referring to the drawings, the back brace 10 has a main board 101, an abdomen pressing board 102, a plurality of positioning members 105 for connecting the main board 101 and the abdomen pressing board 102, and a positioning base board 103 disposed on the main board 101. The main board 101 has a support portion 1011, two hollow portions 1012, 1013 formed at two sides of the support portion 1011 for facilitating a surgical operation and an extending portion 104 integrally formed with the support portion 1011. The positioning base board 103 is disposed on the support portion 1011 and has a plurality of positioning-assisting units 1031. Each of the positioning members 105 has one end connecting an edge of the main board 101 and the other end connecting an edge of the abdomen pressing board 102. As such, the main board 101 and the abdomen pressing board 102 are connected through the positioning members 105, and the abdomen pressing board 102 corresponds in position to the main board 101. In the present embodiment, the positioning members 105 are bandages. When the back brace 10 is disposed on the patient, the positioning members 105 increase the stability of the back brace 10 on the patient so as to reduce displacement or shaking. The number of the positioning members 105 is decided according to the practical requirement.

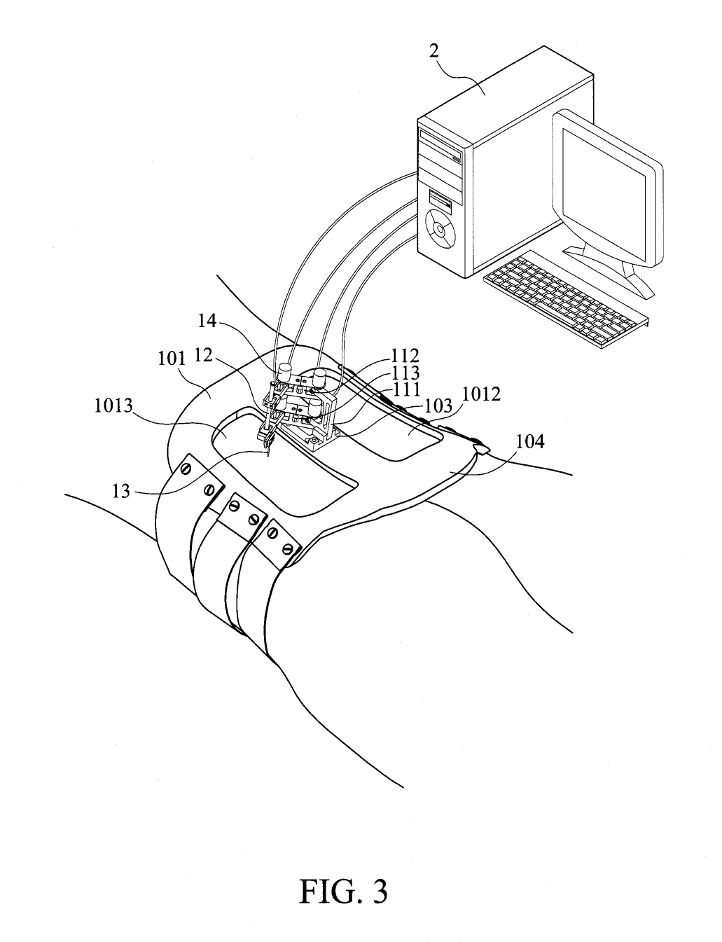

FIG. 3 shows an application of the back brace type surgery positioning apparatus of the present invention. Referring to FIG. 3, the main board 101 of the back brace 10 is disposed on the back of the patient and the abdomen pressing board 102 is disposed correspondingly to the abdomen of the patient. Meanwhile, the extending portion 104 corresponds in position to the sacrum, i.e., the bottom of the spine of the patient. Since the curvature of the human body is relatively larger at the position of the sacrum, the extending portion 104 is designed to comply with the curve of the human body so as to provide a great positioning effect.

After the back brace 10 is disposed on the patient, computed tomography (CT) images of the surgical field of the patient can be captured before the surgical operation. During this process, the positioning-assisting units 1031 of the positioning base board 103 disposed on the support portion 1011 of the back brace 10 can serve as positioning marks to facilitate positioning. In particular, after the CT images are analyzed and processed by a computer device, coordinates and relative positions of the positioning-assisting units 1031 on the patient are generated, thereby determining the position of the positioning base board 103 on the patient. Therefore, automatic positioning is achieved in the present invention.

Subsequently, the positioning mechanism 11 is connected to the positioning base board 103 to perform minimally invasive surgery in the surgical field of the patient. The encoders 14 of the first and second rotating arms 112, 113 can be electrically connected to a computer device 2 so as to transmit displacement data of the first and second rotating arms 112, 113 to the computer device 2 for processing.

During the surgical operation, the operation-assisting member 12 is operated to cause the first and second rotating arms 112, 113 to rotate to a position corresponding to the hollow portions 1012, 1013, and then adjust the direction and position of the surgical tool 13, such that the minimally invasive surgery is performed in the hollow portions 1012, 1013.

During the above-described process, the displacement of the first and second rotating arms 112, 113 is measured by the encoders 14 and the displacement data are further transmitted to the computer device 2 so as to be processed and displayed by the computer device 2.

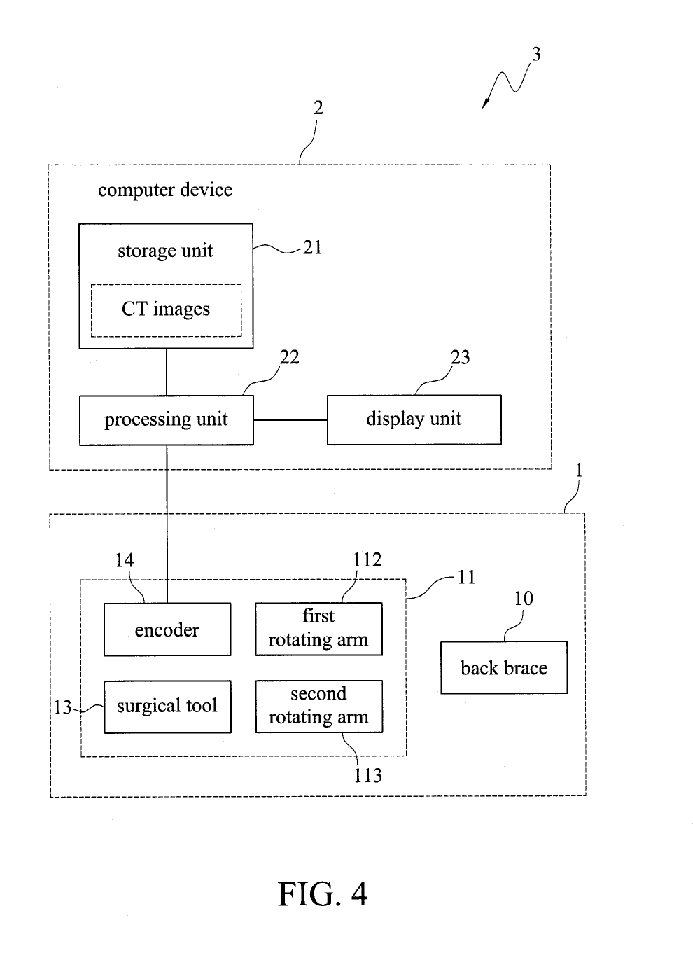

FIG. 4 is a block diagram showing a navigation system 3 of the present invention. Referring to FIG. 4, the system 3 has a back brace type surgery positioning apparatus 1 and a computer device 2. The apparatus 1 has a back brace 10, a positioning mechanism 11 and an operation-assisting member 12. The back brace 10 is configured to be disposed on the back of a patient. The positioning mechanism 11 is disposed on the back brace 10. The positioning mechanism 11 has a first rotating arm 112 and a second rotating arm 113. The first and second rotating arms 112, 113 have a plurality of encoders 14 for measuring displacement of the first and second rotating arms 112, 113, and a surgical tool 13 is further fastened by the second rotating arm 113. The computer device 2 is electrically connected to the encoders 14 to receive displacement data from the encoders 14 and process the displacement data. The computer device 2 has a storage unit 21, a processing unit 22 and a display unit 23.

In practice, before a surgical operation, the back brace 10 is disposed on the back of a patient and CT images are captured. The positioning-assisting units 1031 of the positioning base board 103 on the back brace 10 serve as positioning marks such that after the CT images are analyzed and processed by the processing unit 22 of the computer device 2, coordinates and positions of the positioning-assisting units 1031 are generated. As such, after the support rack 111 of the positioning mechanism 11 is connected to the positioning base board 103, the position of the positioning mechanism 11 relative to the patient is determined through the coordinates and the positions of the positioning-assisting units 1031. The processed CT images are then stored in the storage unit 21.

During the surgical operation, the first and second rotating arms 112, 113 of the positioning mechanism 11 are operated to rotate, and the displacement of the first and second rotating arms 112, 113 is measured by the encoders 14 and transmitted to the computer device 2. The displacement data are then processed by the processing unit 22 and integrated with the CT images from the storage unit 21 and further transmitted to the display unit 23 for display. As such, surgeons can know the position of the surgical tool 13 relative to the patient through the image displayed on the display unit 23 and adjust the position of the surgical tool 13 according to the displayed image.

The above-described descriptions of the detailed embodiments are only to illustrate the preferred implementation according to the present invention, and it is not to limit the scope of the present invention. Accordingly, all modifications and variations completed by those with ordinary skill in the art should fall within the scope of present invention defined by the appended claims.

What is claimed is:

1. A back brace type surgery positioning apparatus, comprising:
a back brace comprising a main board, an abdomen pressing board, a plurality of positioning members for connecting the main board and the abdomen pressing board, and a positioning base board disposed on the main board, wherein the main board has a support portion, an extending portion integrally formed with the support portion, and hollow portions formed at two sides of the support portion for facilitating a surgical operation, and the positioning base board is disposed on the support portion of the main board and has a plurality of positioning-assisting units;
a positioning mechanism connected to the positioning base board, wherein the positioning mechanism comprises a support rack, and first and second rotating arms pivotally mounted to and rotatable with respect to the support rack and provided with a plurality of encoders for measuring displacement of the first and second rotating arms, and the second rotating arm is configured to secure a surgical tool in place; and
an operation-assisting member fastened by the first and second rotating arms and configured to adjust a direction and a position of the surgical tool.

2. The back brace type surgery positioning apparatus of claim 1, wherein the main board is configured to be disposed on a back of a patient and the abdomen pressing board is configured to be disposed on an abdomen of the patient, and the extending portion is corresponding in position to a sacrum of the patient.

3. The back brace type surgery positioning apparatus of claim 1, wherein the first rotating arm has a first fastening member and the second rotating arm has a second fastening member such that the operation-assisting member is fastened by the first and second fastening members and the surgical tool is fastened by the second fastening member.

4. A navigation system, comprising:
a back brace type surgery positioning apparatus, comprising:
a back brace comprising a main board, an abdomen pressing board, a plurality of positioning members for connecting the main board and the abdomen pressing board, and a positioning base board disposed on the main board, wherein the main board has a support portion, an extending portion integrally formed with the support portion, and hollow portions formed at two sides of the support portion for facilitating a surgical operation, and the positioning base board is disposed on the support portion of the main board and has a plurality of positioning-assisting units;
a positioning mechanism connected to the positioning base board, wherein the positioning mechanism comprises a support rack, and first and second rotating arms pivotally mounted to and rotatable with respect to the support rack and provided with a plurality of encoders for measuring displacement of the first and second rotating arms, and the second rotating arm is configured to secure a surgical tool in place; and
an operation-assisting member fastened by the first and second rotating arms and configured to adjust a direction and a position of the surgical tool; and
a computer device configured to store a plurality of computed tomography (CT) images of a back of a patient and electrically connected to the plurality of encoders for receiving displacement data from the encoders and integrating the displacement data with the CT images to thereby display the direction and position of the surgical tool.

5. The navigation system of claim 4, wherein the computer device comprises a storage unit, a processing unit and a display unit, wherein the storage unit is configured to store the CT images, and the processing unit is electrically connected to the storage unit and the display unit and configured to receive the displacement data from the encoders and integrate the displacement data with the CT images and send an integrated result to the display unit for display.

6. The navigation system of claim 4, wherein the main board is disposed on the back of the patient and the abdomen pressing board is disposed on an abdomen of the patient, and the extending portion is corresponding in position to an sacrum of the patient.

7. The navigation system of claim 4, wherein the first rotating arm has a first fastening member and the second rotating arm has a second fastening member such that the operation-assisting member is fastened by the first and second fastening members and the surgical tool is fastened by the second fastening member.
